(19) United States
(12) Patent Application Publication
(10) Pub. No.: US 2024/0013390 A1
(43) Pub. Date: Jan. 11, 2024

(54) IMAGE PROCESSING DEVICE, IMAGE PROCESSING SYSTEM, IMAGE DISPLAY METHOD, AND IMAGE PROCESSING PROGRAM

(71) Applicant: TERUMO KABUSHIKI KAISHA, Tokyo (JP)

(72) Inventors: Katsuhiko SHIMIZU, Fujinomiya-shi (JP); Hiroyuki ISHIHARA, Tokyo (JP); Yasukazu SAKAMOTO, Hiratsuka-shi (JP); Shunsuke YOSHIZAWA, Ebina-shi (JP); Kazuhiro SATOMI, Tokyo (JP); Yoshinao YAZAKI, Tokyo (JP)

(73) Assignee: TERUMO KABUSHIKI KAISHA, Tokyo (JP)

(21) Appl. No.: 18/473,584

(22) Filed: Sep. 25, 2023

Related U.S. Application Data
(63) Continuation of application No. PCT/JP2022/009241, filed on Mar. 3, 2022.

(30) Foreign Application Priority Data
Mar. 25, 2021 (JP) 2021-052439

(51) Int. Cl. G06T 7/00; G06T 7/66; G06V 10/74; G16H 30/20; G16H 30/40

(52) U.S. Cl. CPC G06T 7/0012; G06T 7/66; G06V 10/761; G16H 30/20; G16H 30/40; G06T 2207/10072; G06T 2207/30021; G06T 2207/30204

(57) ABSTRACT

An image processing device, with reference to tomographic data obtained using a sensor moving in a lumen of a biological tissue, displays an image representing the biological tissue on a display, and includes a control unit that acquires specification data specifying at least one location in a space corresponding to the tomographic data, identifies a direction of the at least one location from a centroid in a cross section of the biological tissue orthogonal to a movement direction of the sensor, the direction including the at least one location, as a specification direction, with reference to the tomographic data, identifies a position corresponding to the at least one location in the cross section as a corresponding position according to an identified specification direction and a position of the centroid, and performs control so that a mark is displayed at an identified corresponding position when the image is displayed.

### CROSS-REFERENCES TO RELATED APPLICATIONS

This application is a continuation of International Application No. PCT/JP2022/009241 filed on Mar. 3, 2022, which claims priority to Japanese Application No. 2021-052439 filed on Mar. 25, 2021, the entire content of both of which is incorporated herein by reference.

### TECHNOLOGICAL FIELD

The present disclosure generally relates to an image processing device, an image processing system, an image display method, and an image processing program.

### BACKGROUND DISCUSSION

U.S. Patent Application Publication No. 2010/0215238 A, U.S. Pat. Nos. 6,385,332, and 6,251,072 describe a technique for generating a three-dimensional image of a cardiac cavity or a blood vessel using an ultrasound (US) imaging system.

Treatment using intravascular ultrasound (IVUS) is widely executed for a cardiac cavity, a cardiac blood vessel, a lower limb artery region, and the like. IVUS is a device or a method for providing a two-dimensional image of a plane perpendicular to a longitudinal axis of a catheter.

At present, an operator needs to execute an operation while reconstructing a three-dimensional structure by layering two-dimensional images of IVUS in his/her head, which can be a barrier particularly to young doctors or inexperienced doctors. In order to remove such a barrier, it is conceivable to automatically generate a three-dimensional image representing a structure of a biological tissue such as the cardiac cavity or the blood vessel from the two-dimensional images of IVUS and display the generated three-dimensional (3D) image to the operator.

Recently, a technique for performing electrical interruption by cauterizing the cardiac cavity using an ablation catheter has been widely used. A 3D mapping system in which a position sensor is loaded onto a catheter and a three-dimensional image is drawn using position information when the position sensor touches a myocardial tissue is mainly used in the procedure, but the 3D mapping system can be very expensive and costly. In the case of performing circumferential isolation of the pulmonary vein (PV) or superior vena cave (SVC), there is a need for a marking operation of locations in which cauterization has been performed, but if such an operation can be completed using IVUS, there is a possibility that the cost can be reduced.

In a system for marking at least one location such as a cauterized location of a biological tissue, there is a risk that a display position of a mark may be shifted due to an axial shift or pulsation when the mark is displayed.

### SUMMARY

The present disclosure is to eliminate a shift of marking in a system for marking at least one location related to a biological tissue.

An image processing device as one aspect of the present disclosure is an image processing device that, with reference to tomographic data that is a data set obtained using a sensor moving in a lumen of a biological tissue, displays an image representing the biological tissue on a display, the image processing device including a control unit that acquires specification data specifying at least one location in a space corresponding to the tomographic data, identifies a direction of the at least one location from a centroid in a cross section of the biological tissue orthogonal to a movement direction of the sensor, the direction including the at least one location, as a specification direction, with reference to the tomographic data, identifies a position corresponding to the at least one location in the cross section as a corresponding position according to an identified specification direction and a position of the centroid, and performs control so that a mark is displayed at an identified corresponding position when the image is displayed.

In an embodiment, the control unit detects an inner surface of the biological tissue present in the cross section with reference to the tomographic data, and identifies, as the corresponding position, a position at which a straight line extending in the specification direction from a position of the centroid in the image intersects a detected inner surface.

In an embodiment, the control unit performs control so that the mark is displayed by setting a first region including the corresponding position and a second region around the first region in an inner surface of the biological tissue to different colors in the image.

In an embodiment, the control unit detects an inner surface of the biological tissue present in the cross section with reference to the tomographic data, and identifies, as the corresponding position, a position shifted to a side of the lumen from a position at which a straight line extending in the specification direction from a position of the centroid in the image intersects a detected inner surface.

In an embodiment, the control unit detects an inner surface of the biological tissue present in the cross section with reference to the tomographic data, and identifies, as the corresponding position, a position shifted to an opposite side of the lumen from a position at which a straight line extending in the specification direction from a position of the centroid in the image intersects a detected inner surface.

In an embodiment, the control unit calculates a distance from the centroid in the cross section to the at least one location with reference to the tomographic data, and identifies, as the corresponding position, a position away in the specification direction from a position of the centroid by a calculated distance on a straight line extending in the specification direction from a position of the centroid in the image.

In an embodiment, the control unit acquires the specification data by receiving a user operation of specifying the at least one location on the image.

An image processing system as one aspect of the present disclosure includes the image processing device, and the sensor.

In an embodiment, the image processing system further includes the display.

An image display method as one aspect of the present disclosure is an image display method of, with reference to tomographic data that is a data set obtained using a sensor moving in a lumen of a biological tissue, displaying an image representing the biological tissue on a display, the image display method including, by a computer, acquiring specification data specifying at least one location in a space corresponding to the tomographic data, by the computer, identifying a direction of the at least one location from a centroid in a cross section of the biological tissue orthogonal to a movement direction of the sensor, the direction including the at least one location, as a specification direction, with reference to the tomographic data, by the computer, identifying a position corresponding to the at least one location in the cross section as a corresponding position according to an identified specification direction and a position of the centroid, and, by the computer, performing control so that a mark is displayed at an identified corresponding position when the image is displayed.

An non-transitory computer-readable medium storing an image processing program as one aspect of the present disclosure causes a computer that, with reference to tomographic data that is a data set obtained using a sensor moving in a lumen of a biological tissue, displays an image representing the biological tissue on a display to execute processing including processing of acquiring specification data specifying at least one location in a space corresponding to the tomographic data, processing of identifying a direction of the at least one location from a centroid in a cross section of the biological tissue orthogonal to a movement direction of the sensor, the direction including the at least one location, as a specification direction, with reference to the tomographic data, processing of identifying a position corresponding to the at least one location in the cross section as a corresponding position according to an identified specification direction and a position of the centroid, and processing of performing control so that a mark is displayed at an identified corresponding position when the image is displayed.

According to the present disclosure, a shift of marking can be eliminated in a system for marking at least one location related to a biological tissue.

### BRIEF DESCRIPTION OF THE DRAWINGS

FIG. 1 is a perspective view of an image processing system according to an embodiment of the present disclosure.

FIG. 2 is a diagram illustrating an example of a three-dimensional image and a cross-sectional image displayed on a display by the image processing system according to the embodiment of the present disclosure.

FIG. 3 is a diagram illustrating an example of a cutting region formed by the image processing system according to the embodiment of the present disclosure.

FIG. 4 is a block diagram illustrating a configuration of an image processing device according to the embodiment of the present disclosure.

FIG. 5 is a perspective view of a probe and a drive unit according to the embodiment of the present disclosure.

FIG. 6 is a flowchart illustrating operation of the image processing system according to the embodiment of the present disclosure.

FIG. 7 is a flowchart illustrating operation of the image processing system according to the embodiment of the present disclosure.

FIG. 8 is a diagram illustrating an example of a location specified in the image processing system according to the embodiment of the present disclosure.

FIG. 9 is a diagram illustrating an example of a location specified in the image processing system according to the embodiment of the present disclosure.

FIG. 10 is a diagram illustrating an example of a location specified in the image processing system according to the embodiment of the present disclosure.

FIG. 11 is a diagram illustrating an example of a location specified in the image processing system according to the embodiment of the present disclosure.

FIG. 12 is a flowchart illustrating operation of the image processing system according to the embodiment of the present disclosure.

FIG. 13 is a diagram illustrating a result of binarizing a cross-sectional image of a biological tissue in the embodiment of the present disclosure.

FIG. 14 is a diagram illustrating a result of extracting a point cloud of an inner surface of the biological tissue in the embodiment of the present disclosure.

FIG. 15 is a diagram illustrating a result of calculating centroid positions of a cross section of the biological tissue in the embodiment of the present disclosure.

FIG. 16 is a diagram illustrating a result of calculating centroid positions of a plurality of cross sections of the biological tissue in the embodiment of the present disclosure.

FIG. 17 is a diagram illustrating a result of smoothing the result of FIG. 16.

FIG. 18 is a diagram illustrating an example of a location specified in the image processing system according to the embodiment of the present disclosure.

FIG. 19 is a diagram illustrating an example of a location specified in the image processing system according to the embodiment of the present disclosure.

FIG. 20 is a diagram illustrating an example of marks displayed on the display by the image processing system according to the embodiment of the present disclosure.

FIG. 21 is a flowchart illustrating operation of the image processing system according to the embodiment of the present disclosure.

FIG. 22 is a diagram illustrating an example of a location specified in the image processing system according to a modification of the embodiment of the present disclosure.

FIG. 23 is a diagram illustrating an example of a location specified in the image processing system according to the modification of the embodiment of the present disclosure.

FIG. 24 is a diagram illustrating an example of marks displayed on the display by the image processing system according to the modification of the embodiment of the present disclosure.

FIG. 25 is a diagram illustrating an example of marks displayed on the display by the image processing system according to another modification of the embodiment of the present disclosure.

### DETAILED DESCRIPTION

Set forth below with reference to the accompanying drawings is a detailed description of embodiments of an image processing device, an image processing system, an image display method, and an image processing program.

In the drawings, the same or corresponding portions are denoted by the same reference numerals. In the description of each of the embodiments, description of the same or corresponding parts will be omitted or simplified as appropriate.

An embodiment of the present disclosure will be described.

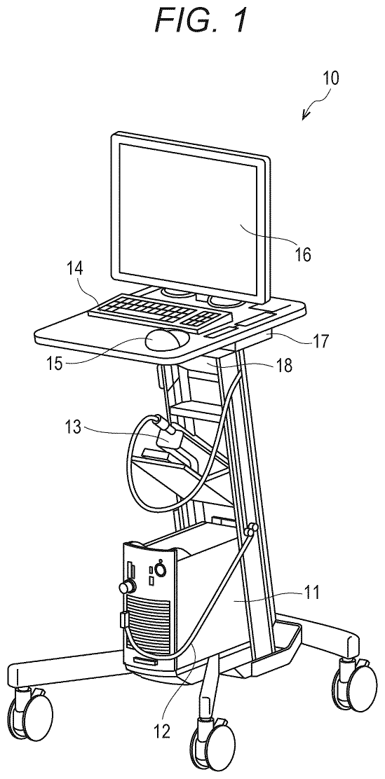

An outline of the present embodiment will be described with reference to FIGS. 1 to 4.

An image processing device 11 according to the present embodiment is a computer that, with reference to tomographic data 51 that is a data set obtained using a sensor moving in a lumen 61 of a biological tissue 60, displays a cross-sectional image 54 representing a cross section 64 of the biological tissue 60 orthogonal to a movement direction of the sensor on a display 16.

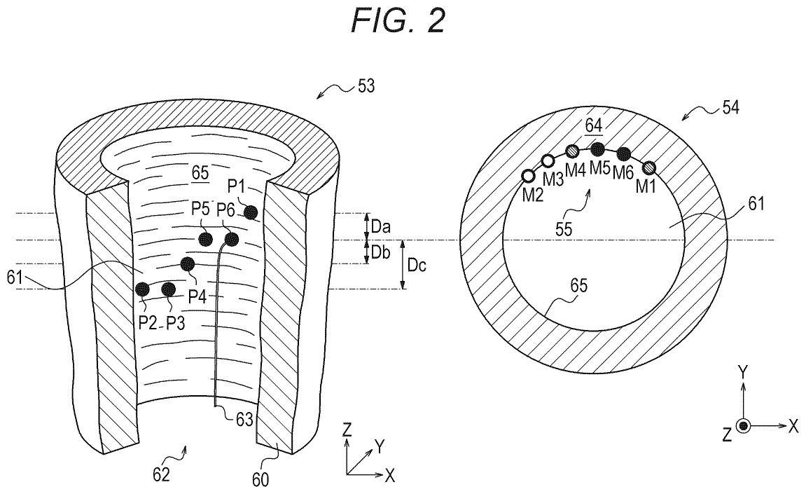

The image processing device 11 acquires specification data specifying at least one location in a space corresponding to the tomographic data 51 as a point Pd. In the example of FIG. 2, six locations cauterized by a catheter 63 on an inner surface 65 of the biological tissue 60 are specified as points P1, P2, P3, P4, P5, and P6.

When the cross-sectional image 54 is displayed, the image processing device 11 performs control so that a mark 55, which varies depending on the distance between the point Pd and the cross section 64 in the movement direction of the sensor is displayed at a position corresponding to the point Pd in the cross-sectional image 54. The position corresponding to the point Pd in the cross-sectional image 54 is a position obtained by shifting the point Pd to the same position as the cross section 64 in the movement direction of the sensor. In the example of FIG. 2, marks M1, M2, M3, M4, M5, and M6 are displayed at positions corresponding to points P1, P2, P3, P4, P5, and P6 in the cross-sectional image 54, respectively. The marks M5 and M6 are displayed in the darkest color since the points P5 and P6 are present in the cross section 64. The mark M4 is displayed in a lighter color than the marks M5 and M6 because the point P4 is away from the cross section 64 by a distance Db in the movement direction of the sensor. The marks M2 and M3 are displayed in the lightest color since the points P2 and P3 are away from the cross section 64 by a distance Dc in the movement direction of the sensor and the distance Dc is longer than the distance Db. The mark M1 is displayed in the same color as the mark M4 since the point P1 is away from the cross section 64 by a distance Da in the movement direction of the sensor and the distance Da is equal to the distance Db.

According to the present embodiment, in a system for marking at least one location in the space corresponding to the tomographic data 51, the relative position of the location in the movement direction of the sensor can be intuitively understood by the user. Therefore, usability of the system can be improved.

In the present embodiment, the image processing device 11 generates and updates three-dimensional data 52 representing the biological tissue 60 with reference to the tomographic data 51 that is a data set obtained using the sensor. The image processing device 11 displays the three-dimensional data 52 as a three-dimensional image 53 on the display 16 together with the cross-sectional image 54. That is, the image processing device 11 displays the three-dimensional image 53 and the cross-sectional image 54 on the display 16 with reference to the tomographic data 51.

The image processing device 11 can form, in the three-dimensional data 52, an opening 62 for exposing the lumen 61 of the biological tissue 60 in the three-dimensional image 53. In the example of FIG. 2, the opening 62 is formed such that all the points P1, P2, P3, P4, P5, and P6 can be seen. Then, the viewpoint when the three-dimensional image 53 is displayed on the screen can be adjusted according to the position of the opening 62. The viewpoint is a position of a virtual camera arranged in a three-dimensional space.

According to the present embodiment, a part of the structure of the biological tissue 60 is cut out in the three-dimensional image 53, so that the lumen 61 of the biological tissue 60 can be seen.

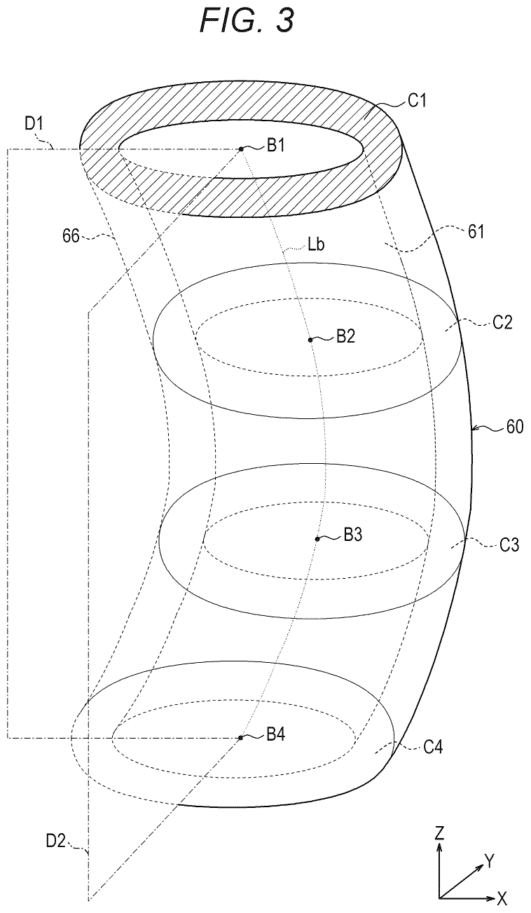

The biological tissue 60 can include, for example, an organ such as a blood vessel or a heart. The biological tissue 60 is not limited to only an anatomically single organ or a part of the anatomically single organ, but also includes a tissue having a lumen across a plurality of organs. An example of such a tissue is, specifically, a part of the vascular tissue extending from the upper part of the inferior vena cava to the lower part of the superior vena cava through the right atrium. In the examples of FIGS. 2 and 3, the biological tissue 60 is a blood vessel.

In FIG. 2, the Z direction corresponds to the movement direction of the sensor, but as illustrated in FIG. 3, for convenience, the Z direction may be regarded as corresponding to the longitudinal direction of the lumen 61 of the biological tissue 60. The X direction orthogonal to the Z direction and the Y direction orthogonal to the Z direction and the X direction may be regarded as corresponding to the lateral directions of the lumen 61 of the biological tissue 60.

In the example of FIG. 3, the image processing device 11 calculates the positions of centroids B1, B2, B3, and B4 of cross sections C1, C2, C3, and C4 of the biological tissue 60 using the three-dimensional data 52. The image processing device 11 sets a pair of planes intersecting at a single line Lb passing through the positions of the centroids B1, B2, B3, and B4 as cutting planes D1 and D2. The image processing device 11 forms, in the three-dimensional data 52, a region interposed between the cutting planes D1 and D2 in the three-dimensional image 53 and from which the lumen 61 of the biological tissue 60 is exposed, as a cutting region 66. In the three-dimensional image 53, the opening 62 as illustrated in FIG. 2 is formed by the cutting region 66 being set to be non-displayed or transparent.

In the case of the three-dimensional model of the bent blood vessel as illustrated in FIG. 3, there is a case where the inside of the blood vessel cannot be correctly displayed if the three-dimensional model is cut at one plane to display the lumen 61. In the present embodiment, as illustrated in FIG. 3, by continuously capturing the centroids of the blood vessel, the three-dimensional model can be cut such that the inside of the blood vessel can be reliably displayed.

In FIG. 3, for convenience, four cross sections C1, C2, C3, and C4 are illustrated as a plurality of cross sections of the biological tissue 60 orthogonal to the Z direction, but the number of cross sections serving as calculation targets of the centroid positions is not limited to four, and is preferably the same as the number of cross-sectional images acquired by IVUS.

A configuration of an image processing system 10 according to the present embodiment will be described with reference to FIG. 1.

The image processing system 10 can include the image processing device 11, a cable 12, a drive unit 13, a keyboard 14, a mouse 15, and the display 16.

In the present embodiment, the image processing device 11 can be a dedicated computer specialized for image diagnosis, but may be a general-purpose computer such as a personal computer (PC).

The cable 12 is used to connect the image processing device 11 and the drive unit 13.

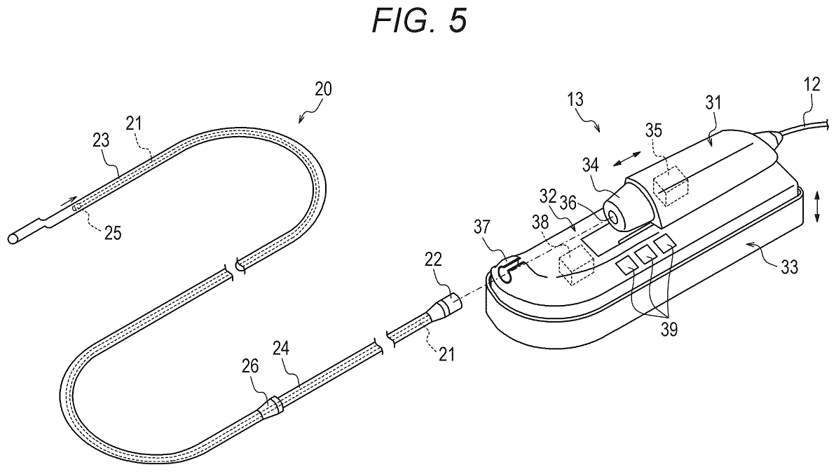

The drive unit 13 is a device that is connected to a probe 20 illustrated in FIG. 5 and drives the probe 20. The drive unit 13 can also be referred to as a motor drive unit (MDU). The probe 20 is applied to IVUS. The probe 20 can also be called an IVUS catheter or an image diagnosis catheter.

The keyboard 14, the mouse 15, and the display 16 are connected to the image processing device 11 via a cable or wirelessly. The display 16 can be, for example, a liquid crystal display (LCD), an organic electro luminescence (EL) display, or a head-mounted display (HMD).

The image processing system 10 optionally further includes a connection terminal 17 and a cart unit 18.

The connection terminal 17 is used to connect the image processing device 11 and an external device. The connection terminal 17 can be, for example, a universal serial bus (USB) terminal. The external device can be, for example, a recording medium such as a magnetic disk drive, a magneto-optical disk drive, or an optical disk drive.

The cart unit 18 can be, for example, a cart with a caster for movement. The image processing device 11, the cable 12, and the drive unit 13 can be installed in the cart body of the cart unit 18. The keyboard 14, the mouse 15, and the display 16 can be installed on the uppermost table of the cart unit 18.

Configurations of the probe 20 and the drive unit 13 according to the present embodiment will be described with reference to FIG. 5.

The probe 20 can include a drive shaft 21, a hub 22, a sheath 23, an outer tube 24, an ultrasound transducer 25, and a relay connector 26.

The drive shaft 21 passes through the sheath 23 inserted into the body cavity of the living body and the outer tube 24 connected to the proximal end of the sheath 23, and extends to the inside of the hub 22 disposed at the proximal end of the probe 20. The drive shaft 21 is rotatably disposed in the sheath 23 and the outer tube 24 with an ultrasound transducer 25 that transmits and receives a signal at the distal end. The relay connector 26 connects the sheath 23 and the outer tube 24.

The hub 22, the drive shaft 21, and the ultrasound transducer 25 are connected to each other to integrally move forward and backward in the axial direction. Therefore, for example, when the hub 22 is pushed toward the distal end, the drive shaft 21 and the ultrasound transducer 25 move toward the distal end inside the sheath 23. For example, when the hub 22 is pulled toward the proximal end, the drive shaft 21 and the ultrasound transducer 25 move toward the proximal end inside the sheath 23 as indicated by arrows.

The drive unit 13 can include a scanner unit 31, a slide unit 32, and a bottom cover 33.

The scanner unit 31 is connected to the image processing device 11 via the cable 12. The scanner unit 31 includes a probe connection portion 34 connected to the probe 20 and a scanner motor 35 as a drive source for rotating the drive shaft 21.

The probe connection portion 34 is detachably connected to the probe 20 via an insertion port 36 of the hub 22 disposed at the proximal end of the probe 20. In the hub 22, the proximal end of the drive shaft 21 is rotatably supported, and the rotational force of the scanner motor 35 is transmitted to the drive shaft 21. In addition, signals are transmitted and received between the drive shaft 21 and the image processing device 11 via the cable 12. In the image processing device 11, generation of a tomographic image of a biological lumen and image processing are performed based on a signal transmitted from the drive shaft 21.

The slide unit 32 is mounted with the scanner unit 31 to be movable forward and backward, and is mechanically and electrically connected to the scanner unit 31. The slide unit 32 can include a probe clamp portion 37, a slide motor 38, and a switch group 39.

The probe clamp portion 37 is disposed coaxially with the probe connection portion 34 at a position distal of the probe connection portion 34, and supports the probe 20 connected to the probe connection portion 34.

The slide motor 38 is a drive source that generates a drive force in the axial direction. The scanner unit 31 moves forward and backward by the drive of the slide motor 38, and the drive shaft 21 moves forward and backward in the axial direction accordingly. The slide motor 38 can be, for example, a servo motor.

The switch group 39 can include, for example, a forward switch and a pull-back switch that are pressed at the time of the forward and backward operation of the scanner unit 31, and a scan switch that is pressed at the time of the start and end of image depiction. The present disclosure is not limited to the examples described in the present disclosure, and various switches are included in the switch group 39, as necessary.

When the forward switch is pressed, the slide motor 38 rotates forward, and the scanner unit 31 moves forward. On the other hand, when the pull-back switch is pressed, the slide motor 38 reversely rotates, and the scanner unit 31 moves backward.

When the scan switch is pressed, image depiction is started, the scanner motor 35 is driven, and the slide motor 38 is driven to move the scanner unit 31 backward. A user such as an operator connects the probe 20 to the scanner unit 31 in advance, and causes the drive shaft 21 to move toward the proximal end in the axial direction while rotating at the start of image depiction. When the scan switch is pressed again, the scanner motor 35 and the slide motor 38 are stopped, and image depiction ends.

The bottom cover 33 covers the entire periphery of the bottom surface and the side surface on the bottom surface side of the slide unit 32, and can approach and separate from the bottom surface of the slide unit 32.

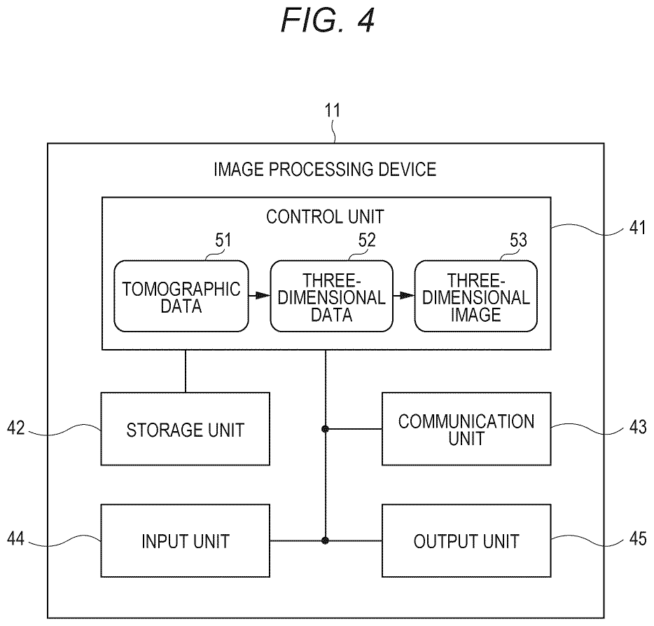

A configuration of the image processing device 11 will be described with reference to FIG. 4.

The image processing device 11 can include a control unit 41, a storage unit 42, a communication unit 43, an input unit 44, and an output unit 45.

The control unit 41 includes at least one processor, at least one programmable circuit, at least one dedicated circuit, or any combination of the least one processor, the at least one programmable circuit, and the at least one dedicated circuit. The processor can be a general-purpose processor such as a central processing unit (CPU) or a graphics processing unit (GPU), or a dedicated processor specialized for specific processing. The programmable circuit can be, for example, a field-programmable gate array (FPGA). The dedicated circuit can be, for example, an application specific integrated circuit (ASIC). The control unit 41 executes processing related to the operation of the image processing device 11 while controlling each unit of the image processing system 10 including the image processing device 11.

The storage unit 42 includes at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 42 can function as, for example, a main storage device, an auxiliary storage device, or a cache memory. The storage unit 42 stores data to be used for the operation of the image processing device 11 such as the tomographic data 51 and data obtained by the operation of the image processing device 11 such as the three-dimensional data 52 and the three-dimensional image 53.

The communication unit 43 includes at least one communication interface. The communication interface can be, for example, a wired local area network (LAN) interface, a wireless LAN interface, or an image diagnosis interface that receives and analog to digital (A/D) converts an IVUS signal. The communication unit 43 receives data to be used for the operation of the image processing device 11 and transmits data obtained by the operation of the image processing device 11. In the present embodiment, the drive unit 13 is connected to the image diagnosis interface included in the communication unit 43.

The input unit 44 includes at least one input interface. The input interface can be, for example, a USB interface, a high-definition multimedia interface (HDMI®) interface, or an interface compatible with near-field communication standard, such as Bluetooth®. The input unit 44 receives a user's operation such as an operation of inputting data to be used for the operation of the image processing device 11. In the present embodiment, the keyboard 14 and the mouse 15 are connected to a USB interface or an interface compatible with near-field communication included in the input unit 44. In a case where the touch screen is disposed integrally with the display 16, the display 16 may be connected to the USB interface or the HDMI interface included in the input unit 44.

The output unit 45 includes at least one output interface. The output interface can be, for example, a USB interface, an HDMI interface, or an interface compatible with near-field communication standard, such as Bluetooth. The output unit 45 outputs data obtained by the operation of the image processing device 11. In the present embodiment, the display 16 is connected to a USB interface or an HDMI interface included in the output unit 45.

A function of the image processing device 11 is implemented by executing an image processing program according to the present embodiment by a processor serving as the control unit 41. That is, the function of the image processing device 11 is implemented by software. The image processing program causes a computer to execute the operation of the image processing device 11 to cause the computer to function as the image processing device 11. That is, the computer functions as the image processing device 11 by executing the operation of the image processing device 11 according to the image processing program.

The program can be stored in a non-transitory computer-readable medium. The non-transitory computer-readable medium can be, for example, a flash memory, a magnetic recording device, an optical disc, a magneto-optical recording medium, or a ROM. The program is distributed, for example, by selling, transferring, or lending a portable medium, such as a secure digital (SD) card, a digital versatile disc (DVD), or a compact disc read only memory (CD-ROM), storing the program. The program may be distributed by storing the program in a storage of a server and transferring the program from the server to another computer. The program may be provided as a program product.

The computer temporarily can store, for example, a program stored in a portable medium or a program transferred from the server in the main storage. Then, the computer reads the program stored in the main storage by the processor and executes processing according to the read program by the processor. The computer may read the program directly from the portable medium and execute the processing according to the program. Each time the program is transferred from a server to a computer, the computer may sequentially execute processing according to the received program. The processing may be executed by what is called an application service provider (ASP) service in which the functions are implemented only by execution instructions and result acquisition instead of the program being transferred from the server to the computer. The program includes information that is provided for processing by an electronic computer and is equivalent to the program. For example, data that is not a direct command to the computer but has a property that defines processing of the computer corresponds to the “information equivalent to the program”.

Some or all of the functions of the image processing device 11 may be implemented by a programmable circuit or a dedicated circuit as the control unit 41. That is, some or all of the functions of the image processing device 11 may be implemented by hardware.

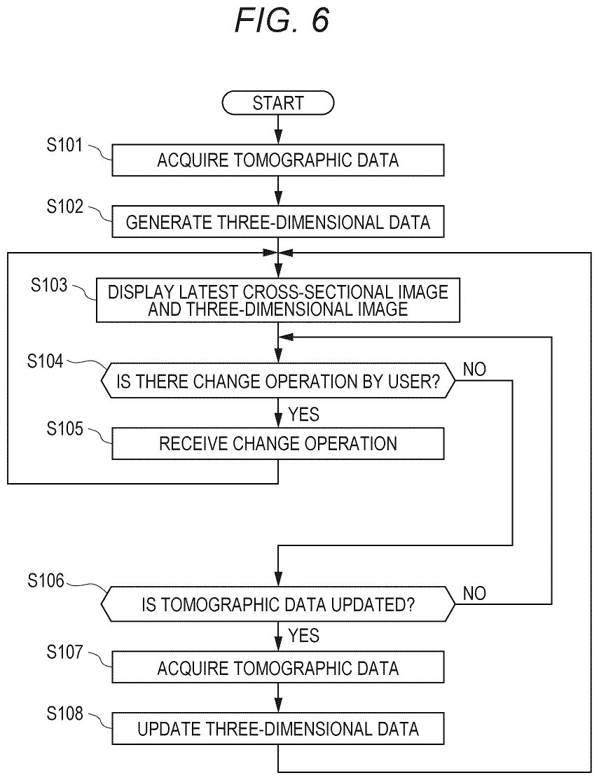

The operation of the image processing system 10 according to the present embodiment will be described with reference to FIG. 6. The operation of the image processing system 10 corresponds to an image display method according to the present embodiment.

Before a start of a flow of FIG. 6, the probe 20 is primed by the user. Thereafter, the probe 20 is fitted into the probe connection portion 34 and the probe clamp portion 37 of the drive unit 13, and is connected and fixed to the drive unit 13. Then, the probe 20 is inserted to a target site in the biological tissue 60 such as a blood vessel or the heart.

In S101, the scan switch included in the switch group 39 is pressed, and the pull-back switch included in the switch group 39 is further pressed, so that a so-called pull-back operation is performed. The probe 20 transmits an ultrasonic wave inside the biological tissue 60 by the ultrasound transducer 25 that moves backward in the axial direction by the pull-back operation. The ultrasound transducer 25 radially transmits the ultrasound wave while moving inside the biological tissue 60. The ultrasound transducer 25 receives a reflected wave of the transmitted ultrasound wave. The probe 20 inputs a signal of the reflected wave received by the ultrasound transducer 25 to the image processing device 11. The control unit 41 of the image processing device 11 processes the input signal to sequentially generate cross-sectional images of the biological tissue 60, thereby acquiring the tomographic data 51, which includes a plurality of cross-sectional images.

Specifically, the probe 20 transmits the ultrasonic wave in a plurality of directions from a rotation center to the outside by the ultrasound transducer 25 while rotating the ultrasound transducer 25 in the circumferential direction and moving the ultrasound transducer 25 in the axial direction inside the biological tissue 60. The probe 20 receives the reflected wave from a reflecting object existing in each of a plurality of directions inside the biological tissue 60 by the ultrasound transducer 25. The probe 20 transmits the signal of the received reflected wave to the image processing device 11 via the drive unit 13 and the cable 12. The communication unit 43 of the image processing device 11 receives the signal transmitted from the probe 20. The communication unit 43 performs A/D conversion on the received signal. The communication unit 43 inputs the A/D converted signal to the control unit 41. The control unit 41 processes the input signal to calculate an intensity value distribution of the reflected wave from the reflecting object existing in the transmission direction of the ultrasonic wave of the ultrasound transducer 25. The control unit 41 sequentially generates two-dimensional images having a luminance value distribution corresponding to the calculated intensity value distribution as the cross-sectional images of the biological tissue 60, thereby acquiring tomographic data 51, which is a data set of the cross-sectional images. The control unit 41 stores the acquired tomographic data 51 in the storage unit 42.

In the present embodiment, the signal of the reflected wave received by the ultrasound transducer 25 corresponds to raw data of the tomographic data 51, and the cross-sectional images generated by processing the signal of the reflected wave by the image processing device 11 correspond to processed data of the tomographic data 51.

In a modification of the present embodiment, the control unit 41 of the image processing device 11 may store the signal input from the probe 20 as it is in the storage unit 42 as the tomographic data 51. Alternatively, the control unit 41 may store data indicating the intensity value distribution of the reflected wave calculated by processing the signal input from the probe 20 in the storage unit 42 as the tomographic data 51. That is, the tomographic data 51 is not limited to the data set of the cross-sectional images of the biological tissue 60, and may be data representing a cross section of the biological tissue 60 at each movement position of the ultrasound transducer 25 in any format.

In a modification of the present embodiment, an ultrasound transducer that transmits the ultrasound wave in the plurality of directions without rotating may be used instead of the ultrasound transducer 25 that transmits the ultrasound wave in the plurality of directions while rotating in the circumferential direction.

In a modification of the present embodiment, the tomographic data 51 may be acquired using optical frequency domain imaging (OFDI) or optical coherence tomography (OCT) instead of being acquired using IVUS. In a case where OFDI or OCT is used, as a sensor that acquires the tomographic data 51 while moving in the lumen 61 of the biological tissue 60, a sensor that acquires the tomographic data 51 by emitting light in the lumen 61 of the biological tissue 60 is used instead of the ultrasound transducer 25 that acquires the tomographic data 51 by transmitting the ultrasound wave in the lumen 61 of the biological tissue 60.

In a modification of the present embodiment, instead of the image processing device 11 generating the data set of the cross-sectional images of the biological tissue 60, another device may generate a similar data set, and the image processing device 11 may acquire the data set from the another device. That is, instead of the control unit 41 of the image processing device 11 processing the IVUS signal to generate the cross-sectional images of the biological tissue 60, another device may process the IVUS signal to generate the cross-sectional images of the biological tissue 60 and input the generated cross-sectional images to the image processing device 11.

In S102, the control unit 41 of the image processing device 11 generates the three-dimensional data 52 of the biological tissue 60 based on the tomographic data 51 acquired in S101. That is, the control unit 41 generates the three-dimensional data 52 based on the tomographic data 51 acquired by the sensor. Note that at this time, if already generated three-dimensional data 52 is present, it is preferable to update only data at a location corresponding to the updated tomographic data 51, instead of regenerating all the three-dimensional data 52 from the beginning. Accordingly, a data processing amount when generating the three-dimensional data 52 can be reduced, and a real-time property of the three-dimensional image 53 in the subsequent S103 can be improved.

Specifically, the control unit 41 of the image processing device 11 generates the three-dimensional data 52 of the biological tissue 60 by layering the cross-sectional images of the biological tissue 60 included in the tomographic data 51 stored in the storage unit 42 and converting the same into three-dimensional data. As a method of three-dimensional conversion, any method among rendering methods such as surface rendering or volume rendering, and various types of processing associated with the rendering method such as texture mapping including environment mapping and bump mapping can be used. The control unit 41 stores the generated three-dimensional data 52 in the storage unit 42.

In a case where the catheter 63 different from an IVUS catheter, such as an ablation catheter, is inserted into the biological tissue 60, the tomographic data 51 includes data of the catheter 63, similarly to the data of the biological tissue 60. Therefore, in S102, the three-dimensional data 52 generated by the control unit 41 also includes the data of the catheter 63 similarly to the data of the biological tissue 60.

The control unit 41 of the image processing device 11 classifies a pixel group of the cross-sectional images included in the tomographic data 51 acquired in S101 into two or more classes. These two or more classes include at least a class of “tissue” to which the biological tissue 60 belongs and a class of “catheter” to which the catheter 63 belongs, and may further include a class of “blood cell”, a class of “medical instrument” other than “catheter” such as a guide wire, a class of “indwelling object” of an indwelling stent or the like, or a class of “lesion” of lime, plaque, or the like. As a classification method, any method may be used, but in the present embodiment, a method of classifying a pixel group of the cross-sectional images by a trained model can be used. The trained model is trained such that a region corresponding to each class can be detected from a cross-sectional image of IVUS as a sample by performing machine learning in advance.

In S103, the control unit 41 of the image processing device 11 displays the three-dimensional data 52 generated in S102 on the display 16, as the three-dimensional image 53. The control unit 41 may set an angle for displaying the three-dimensional image 53 to any angle. The control unit 41 displays the latest cross-sectional image 54 included in the tomographic data 51 acquired in S101 on the display 16 together with the three-dimensional image 53.

Specifically, the control unit 41 of the image processing device 11 generates the three-dimensional image 53 based on the three-dimensional data 52 stored in the storage unit 42. The control unit 41 displays the latest cross-sectional image 54 among the cross-sectional images of the biological tissue 60 included in the tomographic data 51 stored in the storage unit 42 and the generated three-dimensional image 53 on the display 16 via the output unit 45.

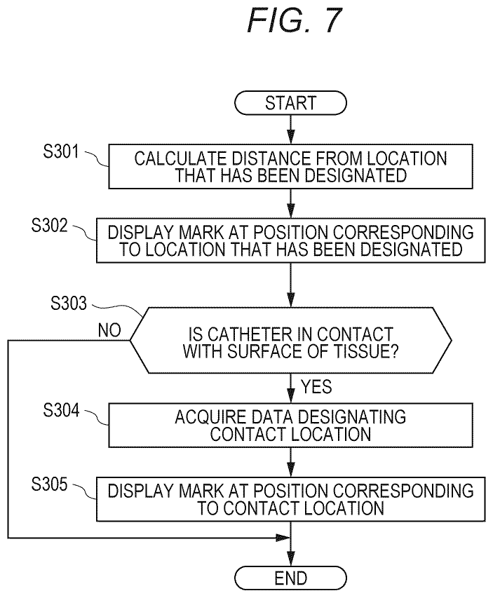

A procedure of processing further executed in S103 will be described with reference to FIG. 7.

In a case where at least one location in the space corresponding to the tomographic data 51 has been specified as the point Pd, processing of S301 and S302 is executed. In a case where there is no specified location, the processing of 301 and S302 is skipped.

In S301, the control unit 41 of the image processing device 11 calculates a distance from the point Pd to the cross section 64 of the biological tissue 60 represented by the cross-sectional image 54 in the movement direction of the sensor.

In S302, the control unit 41 of the image processing device 11 performs control so that a mark 55, which varies depending on the distance calculated in S301 is displayed at a position corresponding to the point Pd in the cross-sectional image 54. In the present embodiment, the control unit 41 changes the color of the mark 55 according to the calculated distance, but may change brightness, transmittance, a pattern, a size, a shape, a direction, or any combination of the brightness, the transmittance, the pattern, the size, the shape, and the direction together with or instead of the color. For example, if the point Pd is close to the cross section 64, the mark 55 may be made larger, and if the point Pd is away from the cross section 64, the mark 55 may be made smaller. Alternatively, the mark 55 may have a rectangular shape if the point Pd is present in the cross section 64, or the mark 55 may have a shape other than a rectangle such as a circle if the point Pd is present in another cross section. Alternatively, the mark 55 may be surrounded by a white frame or may flicker if the point Pd is present in the cross section 64. According to these examples, it is possible to clarify how far the past cauterization position is away and which angular direction is already cauterized in one screen.

In the example of FIG. 2, assuming the points P1, P2, P3, P4, and P5 have been already specified, the control unit 41 of the image processing device 11 calculates the distance Da for the point P1, the distance Dc for the points P2 and P3, the distance Db for the point P4, and a distance 0 for the point P5. Assuming that the distance Da is equal to the distance Db and the distance Dc is longer than the distance Db, the control unit 41 performs control so that the mark M5 in the darkest color is displayed at the position corresponding to the point P5 in the cross-sectional image 54, the marks M1 and M4 in a lighter color than the mark M5 are displayed at the positions corresponding to the points P1 and P4 in the cross-sectional image 54, and the marks M2 and M3 in the lightest color are displayed at the positions corresponding to the points P2 and P3 in the cross-sectional image 54.

In a modification of the present embodiment, in the case of performing control so that the mark 55 is displayed, the control unit 41 of the image processing device 11 may further perform control so that the distance between the point Pd and the cross section 64 in the movement direction of the sensor can be displayed. The unit of the displayed distance can be, for example, a millimeter. In the example of FIG. 2, the control unit 41 may perform control so that the distance Da is displayed next to the mark M1, the distance Dc is displayed next to the marks M2 and M3, and the distance Db is displayed next to the mark M4.

In a modification of the present embodiment, the control unit 41 of the image processing device 11 may hide the mark 55 in a case where the distance between the point Pd and the cross section 64 in the movement direction of the sensor exceeds a threshold. In the example of FIG. 2, assuming the threshold is smaller than the distance Dc, the control unit 41 may hide the marks M2 and M3.

In a modification of the present embodiment, the control unit 41 of the image processing device 11 may change the mark 55 depending on whether the point Pd is present in front of or behind the cross section 64 in the movement direction of the sensor. In the example of FIG. 2, the point P1 is present in front of the cross section 64 in the movement direction of the sensor, that is, above the cross section 64. The points P2, P3, and P4 are present behind the cross section 64 in the movement direction of the sensor, that is, under the cross section 64. Therefore, the control unit 41 may set the color, brightness, transmittance, pattern, size, shape, direction, or any combination of the color, the brightness, the transmittance, the pattern, the size, the shape, and the direction of the mark M1 to be different from those of the marks M2, M3, and M4. For example, the mark M1 may be a triangle convex upward, and the marks M2, M3, and M4 may be triangles convex downward. In a case where the color of the mark M1 is set to a color different from those of the marks M2, M3, and M4, the color may be set such that a difference according to the distance can be secured. For example, in a case where the color of the mark M1 is set to red and the colors of the marks M2, M3, and M4 are set to blue, the depth of red of the mark M1 may be set to be about the same as the depth of the blue of the mark M4, and the depth of the blue of the marks M2 and M3 may be set to be lighter than the depth of the blue of the mark M4. According to these examples, it is possible to clarify how far the past cauterization position is in which direction of up and down in one screen.

In a modification of the present embodiment, the control unit 41 of the image processing device 11 may further perform control so that the distance between the catheter 63 and the point Pd is displayed. The unit of the displayed distance can be, for example, a millimeter. The displayed distance is a distance in a three-dimensional space, that is, an actual distance, although the displayed distance may be a distance on a plane. In the example of FIG. 2, the control unit 41 may perform control so that the distance from the catheter 63 to the point P5 is displayed next to the point P5 that is the shortest distance from the catheter 63. The control unit 41 may further perform control so that a mark, which varies from the mark 55 is displayed at a position corresponding to the distal end of the catheter 63 in the cross-sectional image 54.

In S303, the control unit 41 of the image processing device 11 determines whether the catheter 63 is in contact with the inner surface 65 of the biological tissue 60. Specifically, the control unit 41 analyzes the cross-sectional image 54 and detects the biological tissue 60 and the catheter 63 in the cross-sectional image 54. Then, the control unit 41 determines whether the biological tissue 60 and the distal end of the catheter 63 are in contact with each other by measuring the distance between the biological tissue 60 and the distal end of the catheter 63. Alternatively, the control unit 41 analyzes the three-dimensional data 52 and detects the distal end of the catheter 63 included in the three-dimensional data 52. Then, the control unit 41 determines whether the biological tissue 60 and the distal end of the catheter 63 are in contact with each other by measuring the distance between the biological tissue 60 and the distal end of the catheter 63. The control unit 41 may receive, from an external system that determines whether the distal end of the catheter 63 is in contact with the inner surface 65 of the biological tissue 60 using an electrode disposed at the distal end of the catheter 63, an input of position data indicating a position where the distal end of the catheter 63 is in contact via the communication unit 43 or the input unit 44. Then, the control unit 41 may correct the analysis result of the cross-sectional image 54 or the three-dimensional data 52 with reference to the input position data.

The processing of S303 may be executed using artificial intelligence (AI). In a modification of the present embodiment, a human may determine whether the catheter 63 is in contact with the inner surface 65 of the biological tissue 60 instead of executing the processing of S303.

In a case where it is determined that the catheter 63 is in contact with the inner surface 65 of the biological tissue 60, processing of S304 and S305 is executed. In a case where it is determined that the catheter 63 is not in contact with the inner surface 65 of the biological tissue 60, the processing of S304 and S305 is skipped.

In S304, the control unit 41 of the image processing device 11 acquires specification data specifying a location of the inner surface 65 of the biological tissue 60 with which the catheter 63 is in contact as the point Pd. In a case where at least one location in the space corresponding to the tomographic data 51 has been specified as the point Pd before this time point, one location specified as the point Pd is added. In the present embodiment, the control unit 41 receives a user operation of specifying at least one location on the cross-sectional image 54 as the point Pd to acquire data specifying the point Pd as the specification data, but may automatically detect a position at which the distal end of the catheter 63 is in contact as the point Pd in S303 to acquire data specifying the point Pd as the specification data.

In S305, the control unit 41 of the image processing device 11 performs control so that a new mark is displayed at a position corresponding to a location specified by the specification data acquired in S304 in the cross-sectional image 54.

In the example of FIG. 2, assuming that the point P6 is cauterized, the control unit 41 of the image processing device 11 acquires data specifying the point P6 as the specification data. The control unit 41 performs control so that the mark M6 having the same color as the mark M5 is displayed at a position corresponding to the point P6 in the cross-sectional image 54.

In the present embodiment, the control unit 41 of the image processing device 11 displays a new image representing the cross section 64 corresponding to the position of the sensor as the cross-sectional image 54 on the display 16 every time a new data set is obtained using the sensor. Therefore, when the sensor is moved by a pull-back operation, the distance from the point Pd to the cross section 64 in the movement direction of the sensor changes, and the mark 55 also changes with the change in the distance. By checking the change of the mark 55, the user can obtain a feeling that the sensor approaches the point Pd or a feeling that the sensor moves away from the point Pd by the pull-back operation.

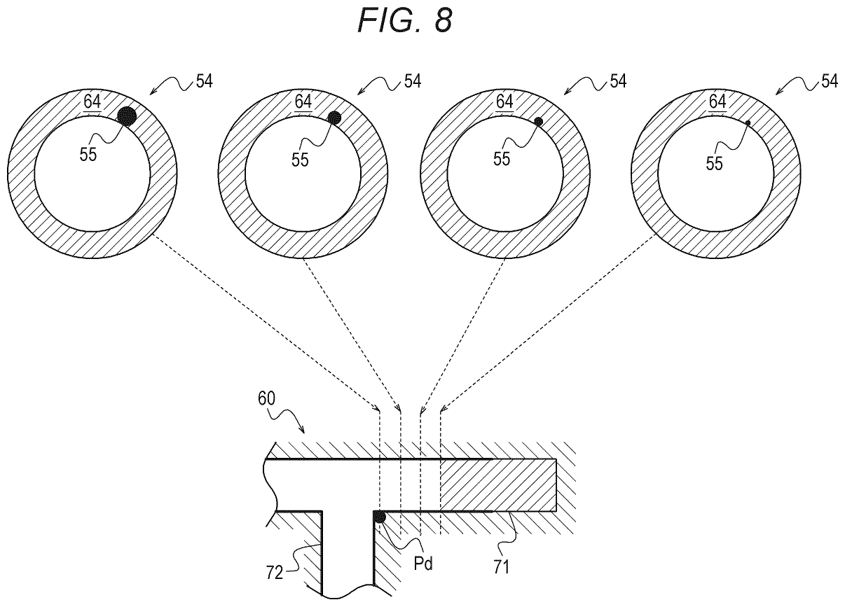

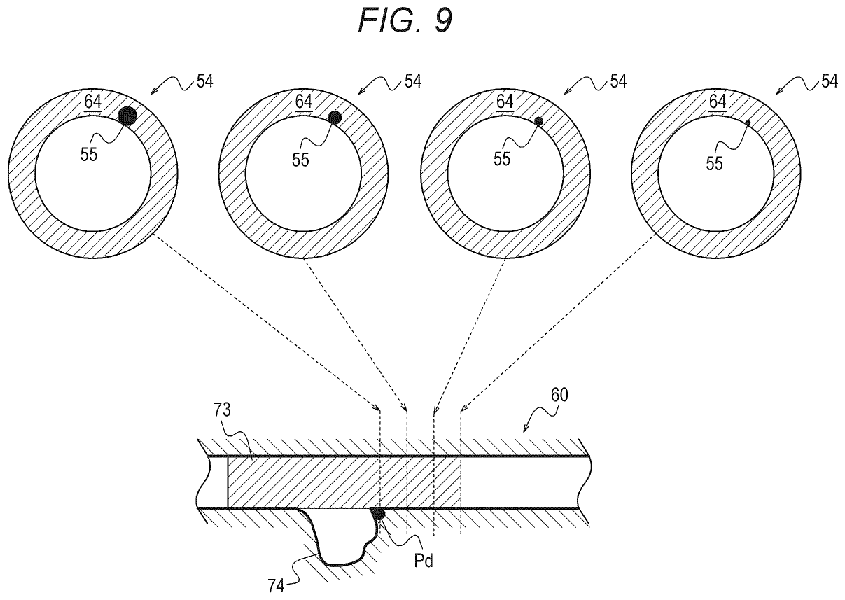

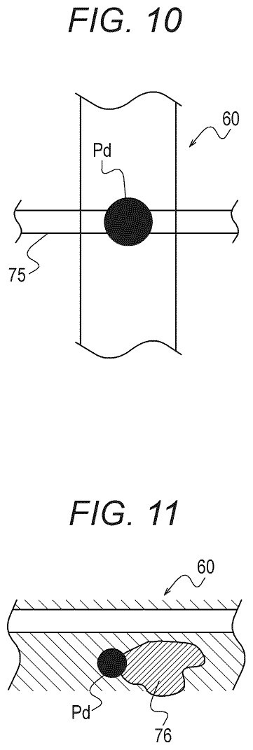

In a modification of the present embodiment, a location other than the location cauterized by the catheter 63 on the inner surface 65 of the biological tissue 60 may be marked. That is, the point Pd is not limited to an ablation point. For example, assuming that the biological tissue 60 is a blood vessel, the root of a branch 72 of a blood vessel may be specified as the point Pd as illustrated in FIG. 8. Alternatively, the root of an aneurysm 74 formed in the blood vessel may be specified as the point Pd as illustrated in FIG. 9. Alternatively, a location intersecting a blood vessel of a nerve 75 may be specified as the point Pd as illustrated in FIG. 10. Alternatively, one location of a tumor 76 formed around a blood vessel may be specified as the point Pd as illustrated in FIG. 11.

In FIG. 8, the upper part illustrates images of respective transverse sections of the blood vessel actually displayed as the cross-sectional images 54 on the display 16, and the lower part is a schematic view of a longitudinal section of the blood vessel. In this schematic diagram, dotted lines each indicate a position of the transverse section as the cross section 64. FIG. 9 is similar to FIG. 8. FIG. 10 is a schematic diagram of a longitudinal section of the blood vessel. FIG. 11 is similar to FIG. 10.

In the examples of FIGS. 8 to 11, it is assumed that the size of the mark 55 changes depending on the distance from the point Pd to the cross section 64 in the movement direction of the sensor. In the example of FIG. 8, if the user desires to place a stent 71 at a certain distance so as not to be placed on the branch 72, the position where the stent 71 should be placed can be rather easily identified by checking the change in size of the mark 55 while performing the pull-back operation. In the example of FIG. 9, if the user desires to place a stent graft 73 over a certain distance so as to cover the aneurysm 74, the position where the stent graft 73 should be placed can be rather easily identified by checking the change in size of the mark 55 while performing the pull-back operation. In a modification, if the user desires to place the stent graft 73 such that the hole of the stent graft 73 is matched with the branch of the blood vessel, the position where the stent graft 73 should be placed can be rather easily identified by checking the change in size of the mark 55 while performing the pull-back operation. According to this example, the distance and direction from the branch of the location where the stent graft 73 is placed can also be easily checked. In the example of FIG. 10, if the user desires to perform ablation while avoiding the nerve 75 intersecting the blood vessel or to perform ablation around the nerve 75, the position where the ablation should be performed can be easily identified by checking the change in size of the mark 55 while performing the pull-back operation. The nerve 75 may be another blood vessel intersecting the blood vessel. In the example of FIG. 11, if the user desires to inject a medicine at a certain distance from the tumor 76 around the blood vessel, the position where the medicine should be injected can be rather easily identified by checking the change in size of the mark 55 while performing the pull-back operation. In any example, the distance from the point Pd to the cross section 64 in the movement direction of the sensor may be indicated by a numerical value. Alternatively, in a case where the distance from the point Pd to the cross section 64 in the movement direction of the sensor is a target distance, the way of displaying the color or the like of the mark 55 may be changed, or the mark 55 may be hidden.

In S104, if there is an operation of setting the angle for displaying the three-dimensional image 53 as a change operation by the user, processing of S105 is executed. If there is no change operation by the user, processing of S106 is executed.

In S105, the control unit 41 of the image processing device 11 receives, via the input unit 44, the operation of setting the angle for displaying the three-dimensional image 53. The control unit 41 adjusts the angle for displaying the three-dimensional image 53 to the set angle. In S103, the control unit 41 displays the three-dimensional image 53 on the display 16 at the angle set in S105.

Specifically, the control unit 41 of the image processing device 11 receives, via the input unit 44, an operation by the user of rotating the three-dimensional image 53 displayed on the display 16 by using the keyboard 14, the mouse 15, or the touch screen disposed integrally with the display 16. The control unit 41 interactively adjusts the angle for displaying the three-dimensional image 53 on the display 16 according to the operation by the user. Alternatively, the control unit 41 receives, via the input unit 44, an operation by the user of inputting a numerical value of the angle for displaying the three-dimensional image 53 by using the keyboard 14, the mouse 15, or the touch screen disposed integrally with the display 16. The control unit 41 adjusts the angle for displaying the three-dimensional image 53 on the display 16 in accordance with the input numerical value.

If the tomographic data 51 is updated in S106, the processing in S107 and S108 is executed. If the tomographic data 51 is not updated, the presence or absence of the change operation by the user is confirmed again in S104.

In S107, similarly to the processing in S101, the control unit 41 of the image processing device 11 processes the signal input from the probe 20 to newly generate cross-sectional images 54 of the biological tissue 60, thereby acquiring the tomographic data 51 including at least one new cross-sectional image 54.

In S108, the control unit 41 of the image processing device 11 updates the three-dimensional data 52 of the biological tissue 60 based on the tomographic data 51 acquired in S107. That is, the control unit 41 updates the three-dimensional data 52 based on the tomographic data 51 acquired by the sensor. Then, in S103, the control unit 41 displays the three-dimensional data 52 updated in S108 on the display 16, as the three-dimensional image 53. The control unit 41 displays the latest cross-sectional image 54 included in the tomographic data 51 acquired in S107 on the display 16 together with the three-dimensional image 53. In S108, it is preferable to update only data at a location corresponding to the updated tomographic data 51. Accordingly, the data processing amount when generating the three-dimensional data 52 can be reduced, and the real-time property of the three-dimensional image 53 can be improved in S108.

As described above, in the present embodiment, the control unit 41 of the image processing device 11, with reference to the tomographic data 51 that is a data set obtained using the sensor moving in the lumen 61 of the biological tissue 60, displays the cross-sectional image 54 representing the cross section 64 of the biological tissue 60 orthogonal to the movement direction of the sensor on the display 16. The control unit 41 acquires specification data specifying at least one location in a space corresponding to the tomographic data 51 as the point Pd. When the cross-sectional image 54 is displayed, the control unit 41 performs control so that the mark 55, which varies depending on the distance between the point Pd and the cross section 64 in the movement direction of the sensor is displayed at a position corresponding to the point Pd in the cross-sectional image 54. Therefore, according to the present embodiment, usability of a system for marking the point Pd associated with the biological tissue 60 can be improved.

According to the present embodiment, an ablation procedure can be guided and a cauterization point can be marked. By checking an ablation point using the cross-sectional image 54, it is possible to know relatively detailed and accurate information as compared with a case of checking an ablation point using the three-dimensional image 53. There are cases where circumferential isolation is performed obliquely with respect to the axis of an IVUS catheter, rather than coplanar, and all ablation points can be checked even in such cases in the present embodiment. Moreover, it is possible to check whether each of the ablation points is in the cross section 64 represented by the cross-sectional image 54, and if not in the cross section 64, how far each of the ablation points is away.

In the present embodiment, the tomographic data 51 classifies each pixel on an ultrasound image into a class such as “tissue”, “blood cell” or “lumen”, and “catheter” other than an IVUS catheter for each ultrasound image, and can include, as a data set, volume data in which a pixel group is layered in the movement direction of the sensor for each class. This volume data corresponds to voxel information. As the specification data, data indicating the position of the point Pd is also incorporated into the data set as volume data of a class “mark location” separately from a class such as “tissue”, “blood cell” or “lumen”, and “catheter”, and the mark 55 is displayed based on the volume data. As will be described below, in a case where the mark location is adjusted by obtaining a vector from the centroid, instead of the data itself indicating the position of the point Pd as the “mark location” but the vector, that is, data indicating the direction may be incorporated into the data set after the vector calculation.

In the present embodiment, as a method of specifying the point Pd, a method in which a user such as an operator specifies the position of the point Pd on a two-dimensional image is used. For example, a method in which the user clicks the point Pd on the two-dimensional image using the mouse 15 is used. In a modification of the present embodiment, a method in which the user specifies the position of the point Pd on the three-dimensional image 53 may be used. For example, a method in which the user clicks the point Pd on the three-dimensional image 53 using the mouse 15 may be used. Alternatively, a method of automatically specifying a region in contact with the ablation catheter as the point Pd based on information that cauterization has been executed may be used. The information that cauterization has been executed may be manually input to the image processing device 11, or may be input to the image processing device 11 from a device that controls the ablation catheter. In any modification, in a case where one point is specified by two-dimensional coordinates or three-dimensional coordinates, a certain range centered on the specified point is marked as one location. In a case where one range centered on a certain point is specified, the specified range is marked as one location. For example, a circular or spherical pointer may specify a range of a certain size as an ablation point. As the distance between the point Pd and the cross section 64 in the movement direction of the sensor, a distance from the center of the specified range to the cross section 64 may be calculated.

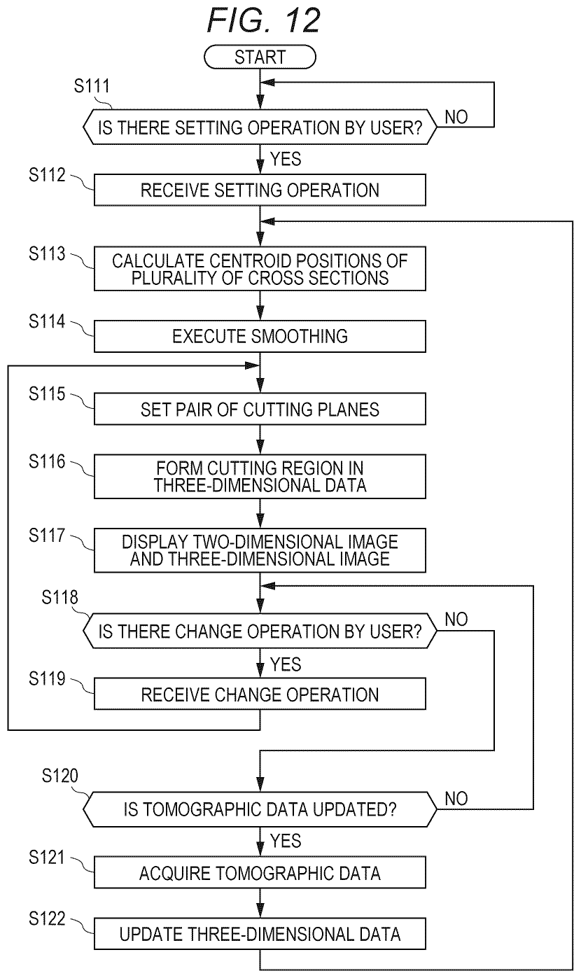

The operation of the image processing system 10 according to the present embodiment will be further described with reference to FIG. 12.

In S111, if there is an operation of setting the cutting region 66 as a setting operation by the user, the processing of S112 is executed.

In S112, the control unit 41 of the image processing device 11 receives, via the input unit 44, the operation of setting the cutting region 66.

In S113, the control unit 41 of the image processing device 11 calculates the centroid positions of the plurality of lateral cross sections of the lumen 61 of the biological tissue 60 by using the latest three-dimensional data 52 stored in the storage unit 42. The latest three-dimensional data 52 is the three-dimensional data 52 generated in S102 if the processing in S108 is not executed, and is the three-dimensional data 52 updated in S108 if the processing in S108 is executed. Note that at this time, if already generated three-dimensional data 52 is present, it is preferable to update only data at a location corresponding to the updated tomographic data 51, instead of regenerating all of the three-dimensional data 52 from the beginning. Accordingly, a data processing amount when generating the three-dimensional data 52 can be reduced, and a real-time property of the three-dimensional image 53 in the subsequent S117 can be improved.

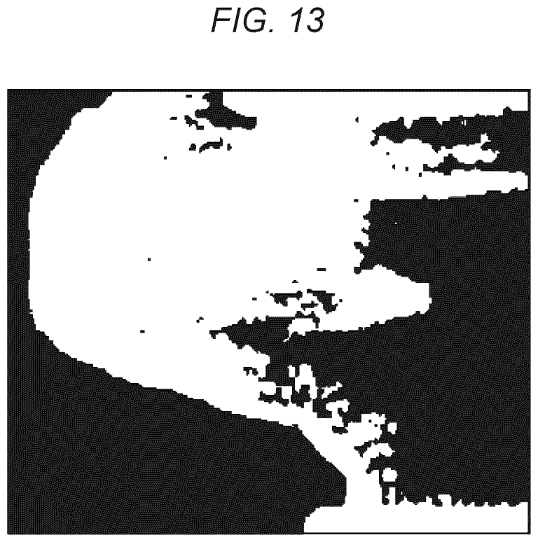

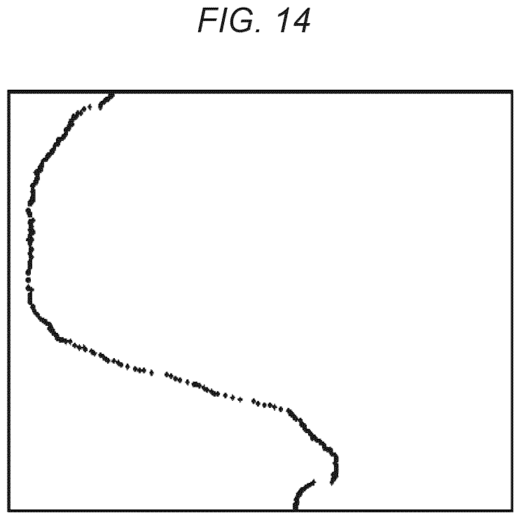

Specifically, as illustrated in FIG. 13, if the control unit 41 of the image processing device 11 generates a corresponding new cross-sectional image 54 in S107 for each of the plurality of cross-sectional images generated in S101, the control unit 41 replaces each of the plurality of cross-sectional images generated in S101 with the new cross-sectional image 54, and then binarizes the cross-sectional image. As illustrated in FIG. 14, the control unit 41 extracts a point cloud on the inner surface 65 of the biological tissue 60 from the binarized cross-sectional image. For example, the control unit 41 extracts a point cloud on an inner surface of a blood vessel by extracting points corresponding to an inner surface of a main blood vessel one by one along a longitudinal direction of the cross-sectional image with an r-axis as a horizontal axis and a 6-axis as a vertical axis. The control unit 41 may simply obtain the centroid of the extracted point cloud on the inner surface, but in that case, since the point cloud is not uniformly sampled over the inner surface, a centroid position shifts. Therefore, in the present embodiment, the control unit 41 calculates the convex hull of the extracted point cloud on the inner surface, and calculates a centroid position Cₙ=(Cₓ, C_{y}) by using a formula for obtaining the centroid of a polygon as follows. However, in the following formula (Mathematical formula 1), it is assumed that n vertices (x₀, y₀), (x₁, y₁), . . . , (xₙ₋₁, yₙ₋₁) are present on the convex hull counterclockwise as the point cloud on the inner surface as illustrated in FIG. 14, and (xₙ, yₙ) is regarded as (x₀, y₀).

$${C}_{x} = \frac{1}{6 A} {{\sum }^{n - 1}}_{i = 0} ( {x}_{i} + {x}_{i + 1} ) ( {x}_{i} {y}_{i + 1} - {x}_{i + 1} {y}_{i} ) \qquad Mathematical formula 1$$

$${C}_{y} = \frac{1}{6 A} {{\sum }_{i = 0}}^{n - 1} ( {y}_{i} + {y}_{i + 1} ) ( {x}_{i} {y}_{i + 1} - {x}_{i + 1} {y}_{i} )$$

$$A = \frac{1}{2} {{\sum }^{n - 1}}_{i = 0} ( {x}_{i} {y}_{i + 1} - {x}_{i + 1} {y}_{í} )$$

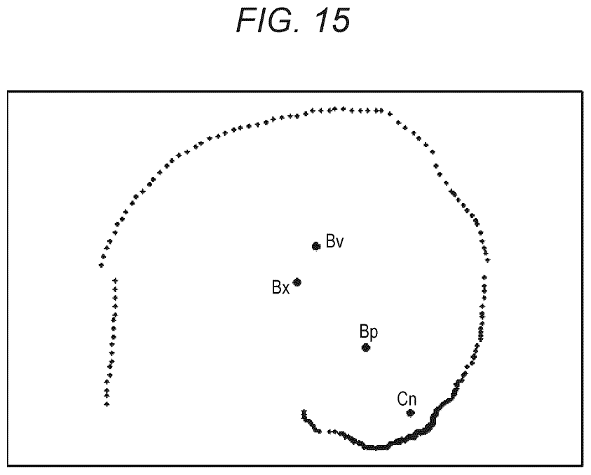

The centroid positions obtained as results are illustrated in FIG. 15. In FIG. 15, a point Cn is the center of the cross-sectional image. A point Bp is a centroid of the point cloud on the inner surface. A point By is a centroid of the vertices of the polygon. A point Bx is a centroid of the polygon serving as a convex hull.

As a method of calculating the centroid position of the blood vessel, a method other than the method of calculating the centroid position of the polygon serving as the convex hull may be used. For example, with respect to an original cross-sectional image that is not binarized, a method of calculating a center position of the maximum circle that falls within the main blood vessel as the centroid position may be used. Alternatively, with respect to the binarized cross-sectional image having the r-axis as the horizontal axis and the θ-axis as the vertical axis, a method of calculating an average position of pixels in a main blood vessel region as the centroid position may be used. The same method as described above may also be used when the biological tissue 60 is not a blood vessel.

In S114, the control unit 41 of the image processing device 11 smooths calculation results of the centroid positions in S113.

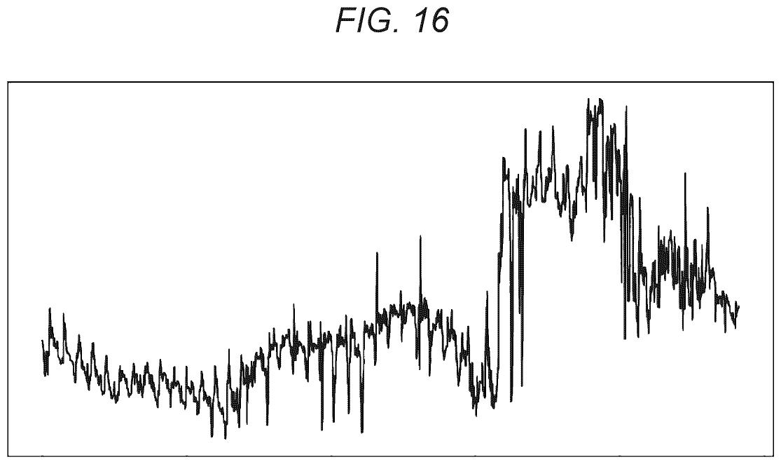

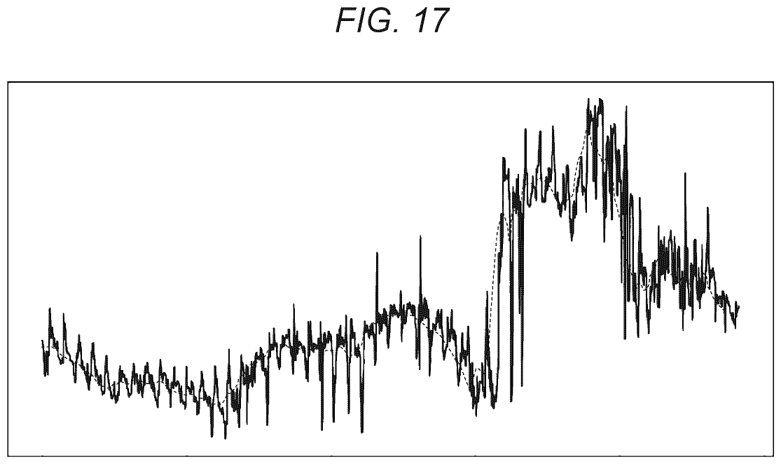

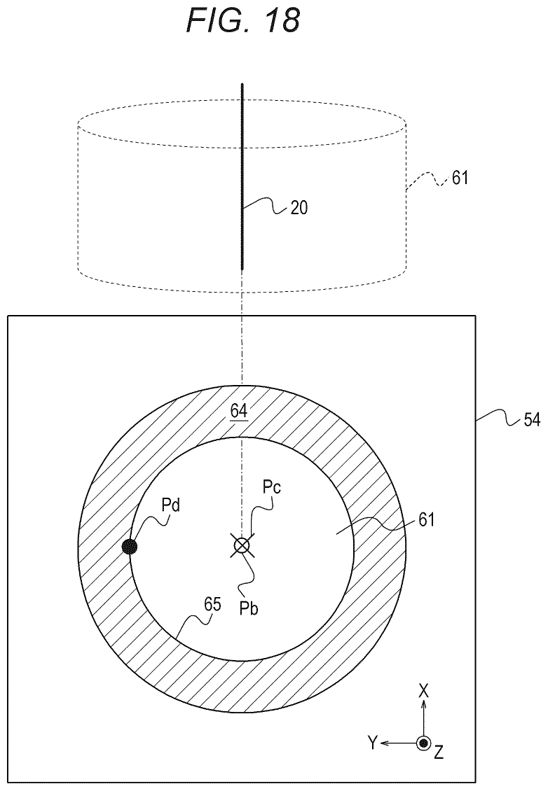

As illustrated in FIG. 16, when the calculation results of the centroid positions are viewed as a time function, it can be seen that an influence of pulsation is relatively large. Therefore, in the present embodiment, the control unit 41 of the image processing device 11 smooths the calculation results of the centroid positions by using movement averages as indicated by a broken line in FIG. 17.

As a smoothing method, a method other than the movement average may be used. For example, exponential smoothing method, kernel method, local regression, Ramer-Douglas-Peucker algorithm, Savitzky-Golay method, smoothed spline, or stretched grid method (SGM) may be used. Alternatively, a method of executing the fast Fourier transform and then removing a high-frequency component may be used. Alternatively, Kalman filter or a low-pass filter such as Butterworth filter, Chebyshev filter, digital filter, elliptical filter, or Kolmogorov-Zurbenko (KZ) filter may be used.

Simple smoothing may cause the centroid positions to enter the tissue. In this case, the control unit 41 may divide the calculation results of the centroid position according to positions of the plurality of cross sections of the biological tissue 60 orthogonal to the Z direction in the Z direction, and may smooth each of the divided calculation results. That is, when a curve of the centroid positions as indicated by the broken line in FIG. 17 overlaps a tissue region, the control unit 41 may divide the curve of the centroid positions into a plurality of sections and execute individual smoothing for each section. Alternatively, the control unit 41 may adjust a degree of smoothing to be executed on the calculation results of the centroid positions according to the positions of the plurality of cross sections of the biological tissue 60 orthogonal to the Z direction in the Z direction. That is, when the curve of the centroid positions as indicated by the broken line in FIG. 17 overlaps the tissue region, the control unit 41 may decrease the degree of smoothing to be executed for a part of the sections including the overlapping points.

In S115, as illustrated in FIG. 3, the control unit 41 of the image processing device 11 can set two planes intersecting at the single line Lb passing through the centroid positions calculated in S113, as cutting planes D1 and D2. In the present embodiment, the control unit 41 smooths the calculation results of the centroid positions in S114, and then sets the cutting planes D1 and D2, but the processing of S114 may be omitted.

Specifically, the control unit 41 of the image processing device 11 can set a curve of the centroid positions obtained as a result of the smoothing in S114 as the line Lb. The control unit 41 sets a pair of planes intersecting at the set line Lb as the cutting planes D1 and D2. The control unit 41 can identify three-dimensional coordinates intersecting with the cutting planes D1 and D2 of the biological tissue 60 in the latest three-dimensional data 52 stored in the storage unit 42 as the three-dimensional coordinates of an edge of the opening 62 exposing the lumen 61 of the biological tissue 60 in the three-dimensional image 53. The control unit 41 stores the identified three-dimensional coordinates in the storage unit 42.

In S116, the control unit 41 of the image processing device 11 forms, in the three-dimensional data 52, a region that is interposed between the cutting planes D1 and D2 in the three-dimensional image 53 and that exposes the lumen 61 of the biological tissue 60, as the cutting region 66.

Specifically, the control unit 41 of the image processing device 11 sets a portion identified by the three-dimensional coordinates stored in the storage unit 42 in the latest three-dimensional data 52 stored in the storage unit 42 to be hidden or transparent when the three-dimensional image 53 is displayed on the display 16. That is, the control unit 41 forms the cutting region 66 set in S112.

In S117, the control unit 41 of the image processing device 11 displays the three-dimensional data 52 in which the cutting region 66 is formed in S116 on the display 16, as the three-dimensional image 53. The control unit 41 displays the cross-sectional image 54 displayed on the display 16 in S103, that is, the two-dimensional image on the display 16 together with the three-dimensional image 53.

Specifically, the control unit 41 of the image processing device 11 generates the three-dimensional image 53 as illustrated in FIG. 2 in which a portion identified by the three-dimensional coordinates stored in the storage unit 42 is hidden or transparent. The control unit 41 displays the latest cross-sectional image 54 among the cross-sectional images of the biological tissue 60 included in the tomographic data 51 stored in the storage unit 42 and the generated three-dimensional image 53 on the display 16 via the output unit 45.

In S117, the processing illustrated in FIG. 7 is further executed similarly to S103.

In S118, if there is an operation of setting the cutting region 66 as a change operation by the user, the processing of S119 is executed. If there is no change operation by the user, processing of S120 is executed.

In S119, the control unit 41 of the image processing device 11 receives, via the input unit 44, the operation of setting the cutting region 66, similarly to the processing in S112. Then, the processing in and after S115 is executed.

If the tomographic data 51 is updated in S120, the processing in S121 and S122 is executed. If the tomographic data 51 is not updated, the presence or absence of the change operation by the user is confirmed again in S118.

In S121, similarly to the processing in S101 or S107, the control unit 41 of the image processing device 11 processes the signal input from the probe 20 to newly generate cross-sectional images 54 of the biological tissue 60, thereby acquiring the tomographic data 51 including at least one new cross-sectional image 54.

In S122, the control unit 41 of the image processing device 11 updates the three-dimensional data 52 of the biological tissue 60 based on the tomographic data 51 acquired in S121. Thereafter, the processing in and after S113 is executed. In S122, it is preferable to update only data at a location corresponding to the updated tomographic data 51. Accordingly, the data processing amount when generating the three-dimensional data 52 can be reduced, and the real-time property of data processing in and after S113.

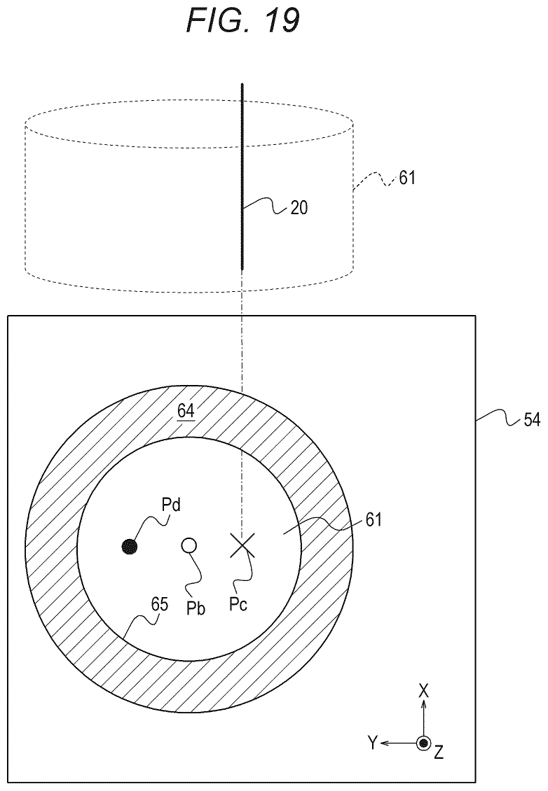

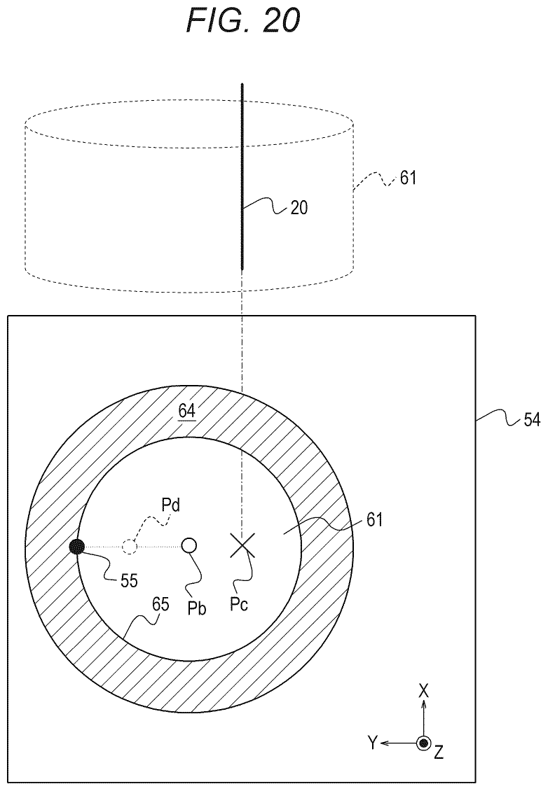

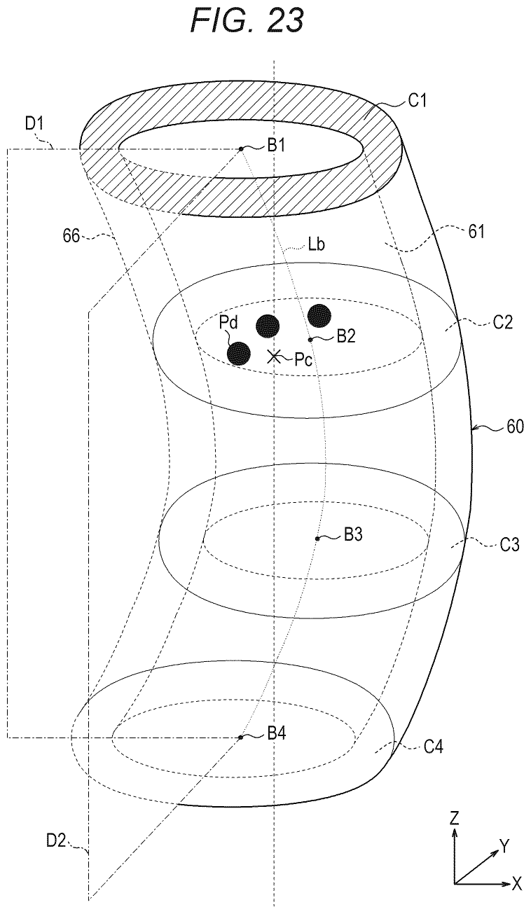

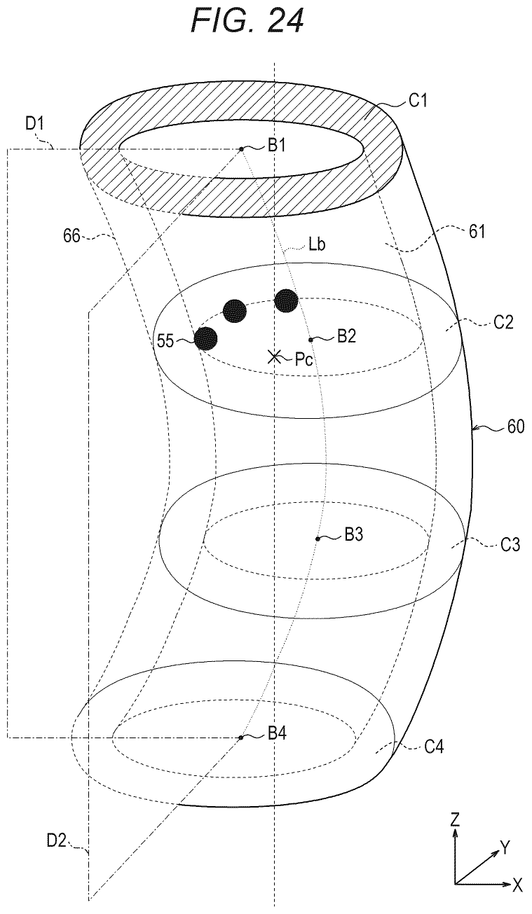

In the present embodiment, marking is performed using a two-dimensional image, but the marking may be performed using the three-dimensional image 53 in a modification of the present embodiment. In a case where the marking is performed using a two-dimensional image, even if the point Pd is marked as illustrated in FIG. 18, the axis of the three-dimensional space is shifted if the axis of the probe 20 is shifted as illustrated in FIG. 19, and the marking becomes meaningless. For example, in FIG. 18, the position of a center Pc of the cross-sectional image 54 matches the position of a centroid Pb of the cross-sectional image 54, but in FIG. 19, the position of the center Pc of the cross-sectional image 54 is shifted (i.e., a considerable amount) from the position of the centroid Pb in the cross-sectional image 54. Therefore, the point Pd is present on the inner surface 65 in FIG. 18, but the point Pd is shifted (i.e., a considerable amount) from the inner surface 65 in FIG. 19. In order to cope with such an issue, in the present embodiment, the mark 55 is displayed at an intersection of a straight line connecting the point Pd and the centroid Pb of the lumen 61 and the inner surface 65 of the biological tissue 60 as illustrated in FIG. 20. Even in a case where the axis of the probe 20 is shifted as illustrated in FIG. 20, the position of the centroid Pb does not change, and the direction from the centroid Pb to the point Pd does not change, so that the shift of marking as illustrated in FIG. 19 can be eliminated. Even if the axis is not shifted, the inner surface 65 sometimes moves due to the influence of pulsation, but even in such a case, the shift of marking can be eliminated. Similarly, in a case where marking is performed using the three-dimensional image 53, even if the point Pd is marked as illustrated in FIG. 22, the position of the point Pd is shifted if the position of the center Pc is shifted as illustrated in FIG. 23. Therefore, for example, the mark 55 is displayed at the relative position from a centroid B2 of a cross section C2 as illustrated in FIG. 24, similarly to the present embodiment, so that the shift of marking as illustrated in FIG. 23 can be eliminated.

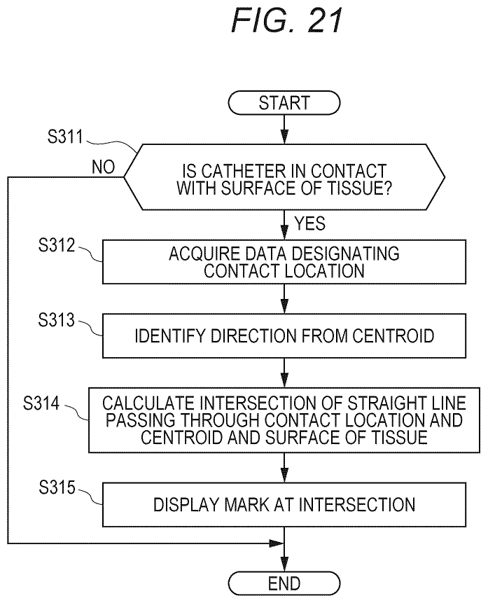

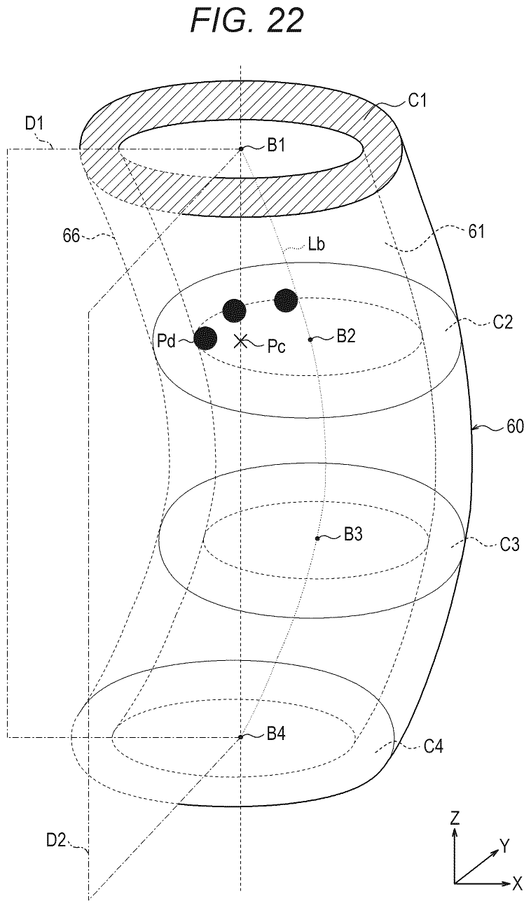

A procedure of marking processing will be described with reference to FIG. 21.

Since processing of S311 is the same as the processing of S303 of FIG. 7, the description of S311 will be omitted.

In a case where it is determined that the catheter 63 is in contact with the inner surface 65 of the biological tissue 60, processing in and after S312 is executed. In a case where it is determined that the catheter 63 is not in contact with the inner surface 65 of the biological tissue 60, the flow of FIG. 21 ends.

In S312, the control unit 41 of the image processing device 11 acquires specification data specifying a location of the inner surface 65 of the biological tissue 60 with which the catheter 63 is in contact as the point Pd, similarly to S304 in FIG. 7. In the present embodiment, the control unit 41 receives a user operation of specifying at least one location on the cross-sectional image 54 as the point Pd to acquire data specifying the point Pd as the specification data, but may automatically detect a position at which the distal end of the catheter 63 is in contact as the point Pd in S311 to acquire data specifying the point Pd as the specification data.

In S313, the control unit 41 of the image processing device 11 identifies the direction of the point Pd specified by the specification data acquired in S304 from the centroid Pb in the cross section 64 as a specification direction with reference the tomographic data 51.

In S314, the control unit 41 of the image processing device 11 identifies the position corresponding to the point Pd in the cross section 64 as a corresponding position according to the specification direction identified in S313 and the position of the centroid Pb. Specifically, the control unit 41 of the image processing device 11 detects the inner surface 65 of the biological tissue 60 present in the cross section 64 with reference to the tomographic data 51. The control unit 41 identifies, as the corresponding position, a position where a straight line extending from the position of the centroid Pb in the cross-sectional image 54 in the specification direction identified in S313 intersects the detected inner surface 65.

In S315, the control unit 41 of the image processing device 11 performs control so that the mark 55 is displayed at the corresponding position identified in S314.

In a modification of the present embodiment, in S314, the control unit 41 of the image processing device 11 may identify, as the corresponding position, a position where a straight line extending in the specification direction identified in S313 from the position of the centroid Pb in the cross-sectional image 54 is shifted toward the lumen 61 from the position intersecting the detected inner surface 65. That is, the mark 55 may be displayed slightly outside the wall from the intersection of the straight line connecting the point Pd and the centroid Pb and the inner surface 65 of the biological tissue 60. According to this modification, it is possible to prevent the edge of the inner surface 65 from being hidden by the mark 55 and information of the edge portion from disappearing. In this modification, the distance between the inner surface 65 and the display position of the mark 55 is stored in the storage unit 42, and is read and applied every time the mark 55 is displayed.

In a modification of the present embodiment, in S314, the control unit 41 of the image processing device 11 may identify, as the corresponding position, a position where a straight line extending in the specification direction identified in S313 from the position of the centroid Pb in the cross-sectional image 54 is shifted to the opposite side of the lumen 61 from the position intersecting the detected inner surface 65. That is, the mark 55 may be displayed slightly inside the wall from the intersection of the straight line connecting the point Pd and the centroid Pb and the inner surface 65 of the biological tissue 60. According to this modification, it is possible to prevent the edge of the inner surface 65 from being hidden by the mark 55 and information of the edge portion from disappearing. In this modification, the distance between the inner surface 65 and the display position of the mark 55 or the distance between the outer surface of the biological tissue 60 and the display position of the mark 55 is stored in the storage unit 42, and is read and applied each time the mark 55 is displayed. Alternatively, the relative display position of the mark 55 between the inner surface 65 and the outer surface is stored in the storage unit 42, and is read and applied each time the mark 55 is displayed. For example, when injecting iPS cells into the wall of a left ventricle, the mark 55 is always displayed in the wall even if the thickness of the wall varies due to pulsation, so that the user can easily identify the position where the cells should be injected.

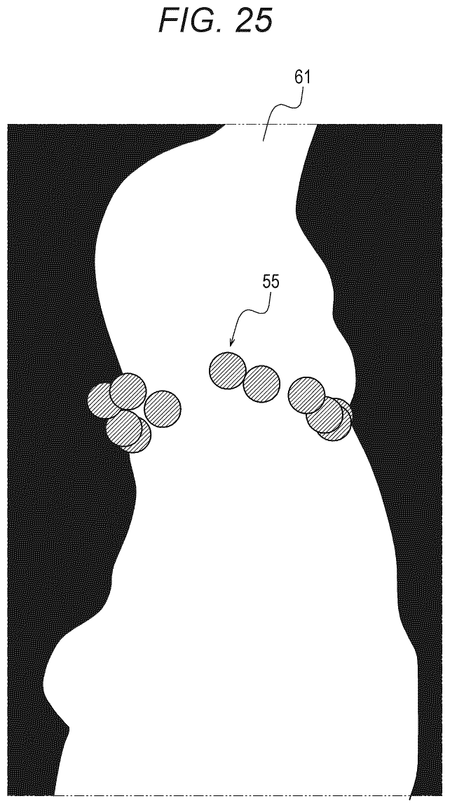

In this modification, as illustrated in FIG. 25, the mark 55 may indicate cauterization positions. In the example of FIG. 25, the lumen 61 of the biological tissue 60 is displayed as a three-dimensional object, and the biological tissue 60 is hidden so that the shape of the lumen 61 can be seen. The outside surface of the three-dimensional object representing the lumen 61 corresponds to the inner surface 65 of the biological tissue 60. The cauterization positions are more easily observed by spheres as the marks 55 being arranged slightly outside the outside surface than in a case where the spheres are arranged on the outside surface or inside the outside surface.

In a modification of the present embodiment, in S314, the control unit 41 of the image processing device 11 may calculate the distance from the centroid Pb to the point Pd in the cross section 64 with reference to the tomographic data 51. On a straight line extending in the specification direction from the position of the centroid Pb in the cross-sectional image 54, the control unit 41 may identify a position away in the specification direction from the position of the centroid Pb by the calculated distance as the corresponding position.

As described above, in the present embodiment, the control unit 41 of the image processing device 11, with reference to the tomographic data 51 that is a data set obtained using the sensor moving in the lumen 61 of the biological tissue 60, displays an image representing the biological tissue 60 on the display 16. The control unit 41 acquires specification data specifying at least one location in a space corresponding to the tomographic data 51 as the point Pd. The control unit 41 identifies the direction of the point Pd from the centroid Pb in the cross section 64 of the biological tissue 60 orthogonal to the movement direction of the sensor including the point Pd as a specification direction with reference to the tomographic data 51. The control unit 41 identifies the position corresponding to the point Pd in the image representing the biological tissue 60 as a corresponding position according to the identified specification direction and the position of the centroid Pb. The control unit 41 performs control so that the mark 55 is displayed at the identified corresponding position when the image representing the biological tissue 60 is displayed. Therefore, according to the present embodiment, the shift of marking can be eliminated in a system for marking the point Pd of the biological tissue 60.

In the present embodiment, the cross-sectional image 54 is used as the “image representing the biological tissue 60”, but the three-dimensional image 53 may be used instead of the cross-sectional image 54. In that case, the control unit 41 of the image processing device 11 may perform control so that the mark 55 is displayed by setting a first region including the corresponding position and a second region around the first region to different colors in the entire inner surface of the biological tissue 60 in the image representing the biological tissue 60. In a case where one point is specified by two-dimensional coordinates or three-dimensional coordinates when the point Pd is specified, a certain range centered on the specified point is marked as the first region. In a case where one range centered on a certain point is specified, the specified range is marked as the first region. For example, a circular or spherical pointer may specify a range of a certain size as the first region.

In the present embodiment, in a case where a plurality of locations of the biological tissue 60 has been specified as the point Pd, the mark 55 is displayed at each of the positions corresponding to the plurality of locations. However, in a modification of the present embodiment, the mark 55 may be displayed only at a location present in the cross section 64 corresponding to the position of the sensor among the plurality of locations. In that case, information of an image in which the mark 55 is set may be stored, and the set mark 55 may be displayed when the same image is displayed as the cross-sectional image 54.

The present disclosure is not limited to the above-described embodiment. For example, two or more blocks described in the block diagrams may be integrated, or one block may be divided. Instead of executing a plurality of steps or processes described in the flowchart in time series according to the description, the steps or processes may be executed in parallel or in a different order according to the processing capability of the device that executes each step or process or as necessary. In addition, modifications can be made within a scope not departing from the gist of the present disclosure.

The detailed description above describes embodiments of an image processing device, an image processing system, an image display method, and an image processing program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing device that, with reference to tomographic data, which is a data set obtained using a sensor moving in a lumen of a biological tissue, displays, on a display, an image representing the biological tissue, the image processing device comprising
a control unit configured to acquire specification data specifying at least one location in a space corresponding to the tomographic data, identify a direction of the at least one location from a centroid in a cross section of the biological tissue orthogonal to a movement direction of the sensor, the direction including the at least one location, as a specification direction, with reference to the tomographic data, identify a position corresponding to the at least one location in the cross section as a corresponding position according to an identified specification direction and a position of the centroid, and perform control so that a mark is displayed at an identified corresponding position when the image is displayed.

2. The image processing device according to claim 1, wherein the control unit is configured to detect an inner surface of the biological tissue present in the cross section with reference to the tomographic data, and identify, as the corresponding position, a position at which a straight line extending in the specification direction from a position of the centroid in the image intersects a detected inner surface.

3. The image processing device according to claim 2, wherein the control unit is configured to perform control so that the mark is displayed by setting a first region including the corresponding position and a second region around the first region in an inner surface of the biological tissue to different colors in the image.

4. The image processing device according to claim 1, wherein the control unit is configured to detect an inner surface of the biological tissue present in the cross section with reference to the tomographic data, and identify, as the corresponding position, a position shifted to a side of the lumen from a position at which a straight line extending in the specification direction from a position of the centroid in the image intersects a detected inner surface.

5. The image processing device according to claim 1, wherein the control unit is configured to detect an inner surface of the biological tissue present in the cross section with reference to the tomographic data, and identify, as the corresponding position, a position shifted to an opposite side of the lumen from a position at which a straight line extending in the specification direction from a position of the centroid in the image intersects a detected inner surface.

6. The image processing device according to claim 1, wherein the control unit is configured to calculate a distance from the centroid in the cross section to the at least one location with reference to the tomographic data, and identify, as the corresponding position, a position away in the specification direction from a position of the centroid by a calculated distance on a straight line extending in the specification direction from a position of the centroid in the image.

7. The image processing device according to claim 1, wherein the control unit is configured to acquire the specification data by receiving a user operation of specifying the at least one location on the image.

8. An image processing system comprising:
the image processing device according to claim 1; and
the sensor.

9. The image processing system according to claim 8, further comprising:
the display.

10. An image display method of, with reference to tomographic data, which is a data set obtained using a sensor moving in a lumen of a biological tissue, displaying an image representing the biological tissue on a display, the image display method comprising:
acquiring, by a computer, specification data specifying at least one location in a space corresponding to the tomographic data;
identifying, by the computer, a direction of the at least one location from a centroid in a cross section of the biological tissue orthogonal to a movement direction of the sensor, the direction including the at least one location, as a specification direction, with reference to the tomographic data;
identifying, by the computer, a position corresponding to the at least one location in the cross section as a corresponding position according to an identified specification direction and a position of the centroid; and
performing, by the computer, control so that a mark is displayed at an identified corresponding position when the image is displayed.

11. The image processing method according to claim 10, further comprising:
detecting, by the computer, an inner surface of the biological tissue present in the cross section with reference to the tomographic data; and
identify, by the computer, as the corresponding position, a position at which a straight line extending in the specification direction from a position of the centroid in the image intersects a detected inner surface.

12. The image processing method according to claim 11, further comprising:
performing, by the computer, control so that the mark is displayed by setting a first region including the corresponding position and a second region around the first region in an inner surface of the biological tissue to different colors in the image.

13. The image processing method according to claim 10, further comprising:
detecting, by the computer, an inner surface of the biological tissue present in the cross section with reference to the tomographic data; and
identifying, by the computer, as the corresponding position, a position shifted to a side of the lumen from a position at which a straight line extending in the specification direction from a position of the centroid in the image intersects a detected inner surface.

14. The image processing method according to claim 10, further comprising:
detecting, by the computer, an inner surface of the biological tissue present in the cross section with reference to the tomographic data; and
identifying, by the computer, as the corresponding position, a position shifted to an opposite side of the lumen from a position at which a straight line extending in the specification direction from a position of the centroid in the image intersects a detected inner surface.

15. The image processing method according to claim 10, further comprising:
calculating, by the computer, a distance from the centroid in the cross section to the at least one location with reference to the tomographic data; and
identifying, by the computer, as the corresponding position, a position away in the specification direction from a position of the centroid by a calculated distance on a straight line extending in the specification direction from a position of the centroid in the image.

16. The image processing method according to claim 10, further comprising:
acquiring, by the computer, the specification data by receiving a user operation of specifying the at least one location on the image.

17. A non-transitory computer-readable medium storing an image processing program that causes a computer that, with reference to tomographic data that is a data set obtained using a sensor moving in a lumen of a biological tissue, displays an image representing the biological tissue on a display to execute a processing comprising:
acquiring specification data specifying at least one location in a space corresponding to the tomographic data;
identifying a direction of the at least one location from a centroid in a cross section of the biological tissue orthogonal to a movement direction of the sensor, the direction including the at least one location, as a specification direction, with reference to the tomographic data;
identifying a position corresponding to the at least one location in the cross section as a corresponding position according to an identified specification direction and a position of the centroid; and
processing of performing control so that a mark is displayed at an identified corresponding position when the image is displayed.

18. The non-transitory computer-readable medium according to claim 17, further comprising:
detecting an inner surface of the biological tissue present in the cross section with reference to the tomographic data;
identify as the corresponding position, a position at which a straight line extending in the specification direction from a position of the centroid in the image intersects a detected inner surface; and
performing control so that the mark is displayed by setting a first region including the corresponding position and a second region around the first region in an inner surface of the biological tissue to different colors in the image.

19. The non-transitory computer-readable medium according to claim 17, further comprising:
detecting an inner surface of the biological tissue present in the cross section with reference to the tomographic data; and
identifying as the corresponding position, a position shifted to a side of the lumen from a position at which a straight line extending in the specification direction from a position of the centroid in the image intersects a detected inner surface.

20. The non-transitory computer-readable medium according to claim 17, further comprising:
detecting an inner surface of the biological tissue present in the cross section with reference to the tomographic data; and
identifying as the corresponding position, a position shifted to an opposite side of the lumen from a position at which a straight line extending in the specification direction from a position of the centroid in the image intersects a detected inner surface.
